(19) United States
(12) Patent Application Publication
(10) Pub. No.: US 2025/0108027 A1
(43) Pub. Date: Apr. 3, 2025

(54) TOPICAL DELIVERY OF EDTA COMPOSITIONS

(71) Applicant: SterileCare Inc.

(72) Inventor: Karen Bernice Mueller, Holland Landing (CA)

(21) Appl. No.: 18/896,413

(22) Filed: Sep. 25, 2024

Related U.S. Application Data
(60) Provisional application No. 63/541,704, filed on Sep. 29, 2023.

(51) Int. Cl. A61K 31/198; A61K 45/06; A61P 31/04

(52) U.S. Cl. CPC A61K 31/198; A61K 45/06; A61P 31/04

(57) ABSTRACT

Improved systems, compositions, and methods of treating infections with antibiotics are provided having reduced risk of creating or promoting drug-resistant microbes or superinfections, where one or more antibiotics susceptible to drug-resistant organisms are topically applied to an infection site in combination with an EDTA solution at elevated pH.

### CROSS-REFERENCE TO RELATED APPLICATIONS

This application claims priority to U.S. Provisional Application No. 63/541,704 titled “TOPICAL DELIVERY OF EDTA COMPOSITIONS” filed Sep. 29, 2023, the entire contents of which are incorporated by reference herein.

### FIELD OF THE INVENTION

This invention pertains to products, methods of manufacture, and methods of use for topical delivery of ethylenediaminetetraacetic acid (EDTA) compositions to wounds or other surfaces of the body for medical purposes such as treating infections.

### BACKGROUND

Antibiotics have long been used in fighting a wide variety of infections or other ailments. However, after prolonged use, drug-resistant microbes have emerged for many once popular antibiotics such as tetracycline, penicillin, etc., making such drugs less useful. For example, streptomycin, discovered in 1943, was the first effective tool for treating tuberculosis and led to a dramatic decline in tuberculosis deaths worldwide. But as early as 1946, physicians at the Mayo Clinic observed strains of bacteria carried by tuberculosis patients that had become roughly 1,000 times as resistant to streptomycin than other strains. Today, streptomycin and related aminoglycosides are threatened with obsolescence due to rising drug resistance in microbes.

In the field of topical application of antibiotics, many challenges are now encountered with existing antibiotics. For example, so treating the skin, drug resistance for neomycin is common in Staphylococcus aureus and other skin microbes. The cyclic lipopeptide polymyxin B faces similar challenges. For mupirocin, increasing resistance among MRSA isolates limits its usefulness. For treating fungal infections of the scalp or toenails with ketoconazole, resistance has been seen in various microbes, including the Malassezia fungi that cause dandruff. Resistance is now seen for fluoroquinolones such as ciprofloxacin to treat eye infections, with resistance rising among Staphylococcus, Streptococcus, and Pseudomonas. Resistance is an increasing issue for Gram-negative bacteria for multiple topical applications with aminoglycosides like tobramycin. For wound care, many antibiotics, ranging from neomycin and polymyxin B to silver sulfadiazine, face challenges from rising drug resistance.

There is a need for improved treatments that can overcome the ongoing challenges of drug-resistant microbes, including reducing the risk of creating drug-resistant microbes when treating an infection or better treating infections from drug-resistant organisms, including multidrug-resistant organisms (MDROs) such as Methicillin-resistant Staphylococcus aureus (MRSA) or multidrug-resistant Mycobacterium tuberculosis (MDR-TB).

The need for improved methods and formulations for using antibiotics without increasing the spread of disease-resistant organisms or promoting the creation of new disease-resistant organisms is motivated by more than just the need to treat bacterial infections. Some antibiotics may have a variety of other uses apart from attacking an infection, but conventional uses still could exacerbate the problem of drug-resistant organisms. For example, fusidic acid has been said to be useful in treating patients with prosthetic joint-related chronic osteomyelitis, while some antibiotics can be useful in treating parasites, such as the antibiotic amphotericin-B in the treatment of leishmaniasis or the antiviral ivermectin as a popular treatment for many parasites. Further, the antibiotic gentamycin can help solve a serious genetic problem in some people by changing how DNA is read, thereby restoring gene function. Generalized severe junctional epidermolysis bullosa (GS-JEB) is an incurable and generally fatal inherited blistering skin disease caused by mutations in the LAMA3, LAMB3, or LAMC2 genes, which typically are nonsense mutations that cause premature termination codons, which impair the production of laminin 332, a protein needed for the epidermal-dermal adherence. As recently reported in Molecular Therapy (https://doi.org/10.1016/j.ymthe.2020.03.006), “Gentamicin induces readthrough of nonsense mutations and restores the full-length protein in various genetic diseases.” However, a challenge is that using an antibiotic to promote the healing of blistering skin could also promote disease-resistant bacteria from repeated antibiotic use, even if the motivation for use is not antimicrobial. Thus, there is a need to improve treatments of many kinds involving antibiotic drugs to reduce the risk of disease-resistant organisms.

There is also a need for improved delivery of pharmaceutical systems in which synergy between a drug and other compounds may be realized, as well as a need for improved systems of treating infection that do not rely on high doses of drugs in the bloodstream. Further, there is a need to apply existing antibiotics in ways that reduce the risk of developing drug-resistant mutants of the targeted microbe and reduce the risk of superinfections from other microbes when treating a patient. A further need is improved methods and compositions for controlling microbial biofilms, which tend to be an increasing factor in many infections, such as ear infections, oral care, wound care, and a variety of skin conditions, such as acne.

The widespread lack of new classes of antibiotics in the past 20 or so years creates a need for improved uses of existing antibiotics, even those that are declining in use due to the rising difficulty of drug resistance and especially multiple drug resistance, and a great need for new approaches to mitigate or contain the problems drug resistance.

In pointing to particular problems or opportunities, unless otherwise indicated, Applicant is not suggesting that any particular aspect claimed herein must necessarily address each or any of the problems or opportunities stated. However, it is believed that various aspects described herein may generally address at least one of the problems or opportunities pointed to, or may instead provide other significant benefits not necessarily specified above.

### SUMMARY

Provided herein is a method for topically treating a treatment site in a subject in need thereof. The method generally includes contacting the treatment site with a pharmaceutical composition comprising a therapeutically effective amount of an antimicrobial susceptible to drug-resistant organisms and a therapeutically effective amount of EDTA, wherein the pharmaceutical composition has a pH from 8.5 to 11, wherein the method has a reduced risk of creating disease-resistant organisms relative to an otherwise identical method without the application of EDTA.

Further provided herein is a method for topically treating a treatment site in a subject in need thereof comprising: contacting the treatment site with a first pharmaceutical composition comprising a therapeutically effective amount of an antimicrobial susceptible to drug-resistant organisms; and contacting the treatment site with a second pharmaceutical composition comprising a therapeutically effective amount of EDTA, wherein the second pharmaceutical composition has a pH from 8.5 to 11, and wherein the method has a reduced risk of creating disease-resistant organisms relative to an otherwise identical method without the application of EDTA.

Further provided herein is a topical pharmaceutical composition effective at reducing a microbial infection from a microbe, the topical pharmaceutical composition comprising an antimicrobial known to be at risk from disease-resistant organisms or at risk of promoting disease-resistant organisms; and at least 0.1% EDTA, wherein the topical therapeutic composition has a pH of 8.5 to 11 and wherein the topical therapeutic composition has a reduced risk of promoting disease-resistant organisms than an otherwise identical composition lacking the EDTA.

Further provided herein is a kit comprising a first container including a first pharmaceutical composition suitable for topical application comprising EDTA at a pH of greater than 8.5; and a second container including a second pharmaceutical composition suitable for topical application comprising an antimicrobial.

### BRIEF DESCRIPTION OF THE DRAWINGS

FIG. 1 depicts a method of manufacturing a medicament comprising EDTA and an antimicrobial.

FIG. 2 depicts a method for applying EDTA and an antimicrobial topically to a treatment site.

### DETAILED DESCRIPTION

Provided herein are pharmaceutical compositions and methods for topically treating a treatment site in a subject in need thereof while reducing the risk of creating disease-resistant organisms. The pharmaceutical compositions generally include EDTA and a therapeutically effective amount of an antimicrobial at a pH greater than 8.5. Also provided are kits that include a first pharmaceutical composition comprising the EDTA at a pH greater than 8.5, and a second pharmaceutical composition comprising an antimicrobial.

In one aspect, a convenient and useful system and method is provided in which a solution comprising EDTA at a pH greater than 8.5, such as from 9 to 11, is provided in combination with a pharmaceutically effective amount of an antimicrobial, wherein the antimicrobial is known to be challenged by drug-resistant microbes, or known to promote the rise of drug-resistant microbes when used conventionally, and wherein synergy between the EDTA and the antimicrobial assists in treating an infection and/or in reducing the risk of disease-resistant organisms. In some aspects, the synergy may be realized through localized, topical delivery to such locations as the skin, open wounds or healing wounds, the eyes, the scalp, nails, the nose or nasal tissues, the mouth or the oral cavity, etc. The subject may include humans or other mammals of any age.

An alkaline EDTA composition with one or more antimicrobials may be applied directly, or the EDTA and the antimicrobial may be applied separately or using separate delivery methods such as an ointment or cream for the antimicrobial and a spray, droplets, emulsion, or a flush for the EDTA solution, or vice versa.

Methods of application, in general, may include manual or mechanical application of a cream or ointment such as an oil/water emulsion, a water/oil emulsion, or a more complex emulsion (oil/water/oil or water/oil/water). It may also include a solution, a colloidal suspension, a slurry, a paste, a spray, an aerosol, droplets, an emulsion, or a flush. Tools for delivery may include a medicated wipe such as a wet wipe, a brush applicator, a medicine dropper, a medicated wound dressing, a spray from a pressurized can or bottle, a squeezable nasal spray container, a spray bottle with a manual trigger, a nebulizer or atomizer, an inhaler, etc.

The combination of EDTA with antimicrobials in the methods and compositions described herein may not only be effective in inhibiting or destroying pathogens, but, in some aspects, may also be effective in mitigating harmful biofilms in the body, such as biofilms in wounds, in or on the skin, in the ears, the oral cavity, etc. Without wishing to be bound by theory, it is believed that EDTA's contribution in mitigating or undermining biofilm in the methods and compositions described herein may involve its ability to sequester certain materials such as calcium, magnesium, zinc, and iron that are needed for bacterial growth and biofilm formation. The ability of EDTA to chelate cations may help remove ions essential for the extracellular polymeric substances that biofilm-formers use to adhere to surfaces and protect a colony of microbes. Such effects may involve interfering with microbial enzymes, quorum signaling, the structure of extracellular polymeric substances, etc. Significant gains in performance may be obtained by combining EDTA, particularly in its tetrasodium form at elevated pH, with selected antibiotics and optionally other agents, such as materials that may provide further synergy with EDTA in undermining biofilm (e.g., N-acetyl cysteine, cysteine salts, ectoin), etc.

The pharmaceutical composition includes EDTA or a pharmaceutically acceptable salt thereof. For example, the EDTA may be tetrasodium EDTA, disodium EDTA, sodium calcium edetate, or a combination thereof.

The EDTA may be present in the pharmaceutical composition in an amount from about 0.1 wt % to about 15 wt %. For example, the EDTA may be present in the pharmaceutical composition in an amount from about 0.1 wt % to about 0.5 wt %, about 0.1 wt % to about 1 wt %, about 0.1 wt % to about 5 wt %, about 0.1 wt % to about 10 wt %, about 0.1 wt % to about 15 wt %, about 0.5 wt % to about 15 wt %, about 1 wt % to about 15 wt %, about 5 wt % to about 15 wt %, about 10 wt % to about 15 wt %, about 1 wt % to about 5 wt %, about 1 wt % to about 10 wt %, or about 5 wt % to about 10 wt %. As another example, the EDTA may be present in the composition in an amount of about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 1 wt2%, about 13 wt %, about 14 wt %, or about 15 wt %. In some embodiments, the EDTA may be present in the pharmaceutical composition in an amount from about 0.2 wt % to about 12 wt %, or from about 0.5 wt % to about 11 wt %.

The pharmaceutical composition may have a pH of about 8.5 or higher. For example, the composition may have a pH of about 8.5 or higher, about 9.0 or higher, about 9.5 or higher, about 10.0 or higher, about 10.5 or higher, about 11.0 or higher, or about 11.5 or higher. As another example, the pharmaceutical composition may have a pH from about 8.5 to about 9.0, about 8.5 to about 9.5, about 8.5 to about 10.0, about 8.5 to about 10.5, about 8.5 to about 11.0, about 8.5 to about 11.5, about 9.0 to about 11.5, about 9.5 to about 11.5, about 10.0 to about 11.5, about 10.5 to about 11.5, about 11.0 to about 11.5, about 9.0 to about 11.0, or about 9.5 to about 10.5. As another example, the composition may have a pH of about 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, or about 11.5.

The pharmaceutical composition may be formulated as a solution, topical ointment, gel, suspension, or other form suitable for topical application to the skin, open wounds or healing wounds, the eyes, the ears, the scalp, nails, the nose or nasal tissues, the sinuses or the mouth or the oral cavity of a subject in need thereof. In a particular aspect, the pharmaceutical composition may be a solution provided for use in generating a spray or aerosol that can be used for directly treating an open wound, a skin ailment, an oral care infection, an eye infection, etc.

The pharmaceutical composition, when formulated as a solution, may also have a physiologically acceptable concentration of sodium chloride, whether isotonic (about 0.9 wt % or from 0.7 wt % to 0.9 wt %), slightly hypotonic (e.g., from 0.6 wt % to 0.75 wt %), or hypertonic (e.g., from 0.9 wt % to 1.3 wt %, 1.5 wt %, 3.5 wt %, 5 wt %, 7 wt %, 9 wt %, or 11 wt %).

Antimicrobials may include antibacterial agents as well as agents effective in treating fungal infections. In some aspects, EDTA coupled with antifungal agents can effectively improve efficacy and reduce the risk of new drug resistant organisms or mitigate drug resistance in targeted or peripheral organisms. The antimicrobial may be polar, lipophilic, both polar and lipophilic, or may have a molecular weight of 450 of greater, 550 or greater, 650 or greater, 750 or greater, 1,000 or greater, 1,500 or greater, or 2000 or greater. Antimicrobials subject to risks from disease-resistant organisms or superinfection may include those from major drug classes such as:
Aminoglycosides, such as amikacin, gentamicin, gramicidin, neomycin, streptomycin, and tobramycin;
Avermectins, such as ivermectin;
Carbapenems, such as doripenem, ertapenem, imipenem, and meropenem;
Cephalosporins, such as cefamandole, cefazolin, cefepime, cefotaxime, ceftriaxone, cephalosporins, and cephalexin;
Cyclic peptides, such as bacitracin, cyclosporine, and ramoplanin;
Dihydrofolate reductase (DHFR) inhibitors, such as trimethoprim, pyrimethamine proguanil, brodimoprim, and iclaprim;
Echinocandins, such as caspofungin, micafungin, and anidulafungin;
Fluoroquinolones, such as ciprofloxacin, levofloxacin, and moxifloxacin;
Fusidanes, such as fusidic acid and sodium fusidate;
Glycopeptides, such as teicoplanin and vancomycin;
Imidazole antifungals: clotrimazole, econazole, ketoconazole, miconazole, and tioconazole.
Lincosamides such asclindamycin;
Lipopeptides and linear peptides, such as daptomycin, iturin, nisin, polymyxin B, colistin (polymyxin E), surfactin, and tyrothricin;
Macrolides, such as azithromycin, clarithromycin, and erythromycin; Monocarboxylic acids, such as mupirocin;
Monobactams, such as aztreonam;
Nitrofurans, such as nitrofurazone, furazolidone, furaltadone, and nitrofurantoin;
Nitroimidazoles, such as metronidazole, tinidazole, nimorazole, dimetridazole, pretomanid, ornidazole, megazol, azanidazole, and benznidazole;
Oxazolidinones, such as linezolid, contezolid, sutezolid, eperezolid, radezolid, posizolid, tedizolid, and delpazolid;
Penicillins, such as amoxicillin, ampicillin, methicillin, and penicillin;
Phenicols, such as chloramphenicol, azidamfenicol, florfenicol, and thiamphenicol; Pleuromutilins, such as tiamulin, valnemulin, and retapamulin;
Polyene antifungals, such as nystatin, natamycin/pimaricin, hamycinand amphotericin B;
Rifamycins, such as rifampin, rifabutin, rifapentine, and rifalazil;
Sulfonamides, such as sulfamethoxazole, sulfonamides, sulfadiazine and silver sulfadiazne, sulfacetamide, sulfasalazine, sulfathiazole, sulfisoxazole, and sulfadoxine;
Sulfones, such as dapsone, sulfanilamide, and sulfapyridine;
Tetracyclines, such as tetracycline, doxycycline, minocycline, tigecycline, eravacycline, and omadacycline;
Triazoles, such as fluconazole, itraconazole, voriconazole, posaconazole, and isavuconazole;
Tuberactinomycins, such asviomycin, enviomycin, and capreomycin;
Others, such as quinupristin, dalfopristin, chlorhexidine, peroxides, silver nanoparticles, ectoin, etc.

These antimicrobials are known to treat a wide variety of skin and other infections for which topical application may be appropriate. A few examples of infections and some examples of useful antimicrobials may include acne (tetracycline, clindamycin, brodimoprim), blepharitis (erythromycin, bacitracin, polymyxin B), cellulitis (fusidic acid), conjunctivitis (sulfamethoxazole, fusidic acid, tobramycin), dermatitis herpetiformis (dapsone), eczema (mupirocin, fusidic acid, clindamycin, metronidazole), folliculitis (mupirocin, clindamycin), fungal infections (numerous antifungals such as the triazoles), Hansen's disease (dapsone), impetigo (mupirocin, fusidic acid, retapamulin), keratitis (moxifloxacin, ciprofloxacin, gentamicin), periodontal disease (tetracycline fibers, doxycycline, minocycline), rosacea (ivermectin), seborrheic dermatitis (sulfacetamide, ketoconazole), skin infections (neomycin, bacitracin), bed sores/pressure ulcers (silver sulfadiazine and nitrofurazone), oral thrush/candidosis (proguanil), etc.

In some aspects, the antimicrobials that are combined with EDTA solutions and applied via aerosol or spray may be relatively poorly absorbed by contact with mucous membranes, wounds, nasal pathways, eyes, the oral cavity, etc., such that application of an effective dose to treat a localized infection such as an infection in the eyes, ears, nose, throat, burn or open wound can be achieved without substantial amounts of the antimicrobial entering the bloodstream. In this way, the antimicrobial concentration, as applied topically, may have little impact on systemic levels of the drug and can be effective in their topical application, remaining in the presence of EDTA solution at elevated pH, thereby maintaining conditions for synergy.

Selection of suitable doses of antimicrobial can be pursued in light of published minimum inhibitory concentration (MIC) data from sources such as the European Committee on Antimicrobial Susceptibility Testing (EUCAST), which publishes documents such as “Breakpoint tables for interpretation of MICs and zone diameters,” Version 13.1, Jun. 29, 2023, available at https://www.eucast.org/clinical_breakpoints. Such charts may list a concentration as, for example, “S<0.03 mg/l” and “R>0.8 mg/l,” meaning that a strain of the bacteria is considered susceptible to the drug when its MIC is less than 0.03 mg/l but is considered resistant when its MIC is greater than 0.8 mg/l. Unless otherwise indicated, references herein to a listed MIC may be taken to refer to the susceptibility breakpoint.

The antimicrobial may be present in an amount from about 0.01 wt % to about 20 wt % on a dry weight basis, such as from about 0.1 wt % to about 10 wt %, or about 0.1 wt % to about 3 wt %. For example, the antimicrobial may be included in the pharmaceutical composition an amount from about 0.01 wt % to about 0.1 wt %, about 0.01 wt % to about 1 wt %, about 0.01 wt % to about 5 wt %, about 0.01 wt % to about 10 wt %, about 0.01 wt % to about 20 wt %, about 0.1 wt % to about 20 wt %, about 1 wt % to about 20 wt %, about 5 wt % to about 20 wt %, about 10 wt % to about 20 wt %, about 0.1 wt % to about 5 wt %, or about 0.1 wt % to about 10 wt %. Those having skill in the art will be capable of determining the appropriate therapeutic dose of the antimicrobial to administer to the patient in need thereof.

The pharmaceutical composition may include a first antimicrobial and a second antimicrobial, wherein the first antimicrobial may be present in an amount from about 0.01 wt % to about 20 wt % on a dry weight basis, such as from about 0.1 wt % to about 10 wt %, or about 0.1 wt % to about 3 wt %; and the second antimicrobial may be present in an amount from about 0.01 wt % to about 20 wt % on a dry weight basis, such as from about 0.1 wt % to about 10 wt %, or about 0.1 wt % to about 3 wt %.

The pharmaceutical composition may further include a pharmaceutically acceptable buffer. The buffer may include an alkaline glycine buffer (e.g., a glycine solution adjusted with NaOH to a pH of 10 or 10.5), a Tris buffer (tris(hydroxymethyl)aminomethane); a carbonate-bicarbonate buffer with sodium carbonate and sodium bicarbonate; or a HEPES buffer (4-(2-hydroxyethyl)-1-piperazineethanesulfonic acid).

The buffer may be present in the pharmaceutical composition in an amount from about 5 wt % to about 60 wt %. For example, the buffer may be present in the pharmaceutical composition in an amount from about 5 wt % to about 10 wt %, about 5 wt % to about 20 wt %, about 5 wt % to about 30 wt %, about 5 wt % to about 40 wt %, about 5 wt % to about 50 wt %, about 5 wt % to about 60 wt %, about 10 wt % to about 60 wt %, about 20 wt % to about 60 wt %, about 30 wt % to about 60 wt %, about 40 wt % to about 60 wt %, or about 50 wt % to about 60 wt %. In other examples, the buffer may be present in the pharmaceutical composition in an amount of about 5 wt %, 10 wt %, 15 wt %, 20 wt %, 25 wt %, 30 wt %, 35 wt %, 40 wt %, 45 wt %, 50 wt %, 55 wt %, or about 60 wt %.

The pharmaceutical composition may further include a solubilizer or surfactant, particularly when the pharmaceutical composition is formulated as a solution, slurry, paste, suspension, or emulsion. The solubilizer or surfactant may include Kolliphor EL (a nonionic solubilizer and emulsifier made by reacting castor oil with ethylene oxide), glycerin, glycerol, isopropylidene glycerol, 1,2 propanediol, 1,3 propanediol, propylene glycol, sodium lauryl sulfate, and the like. The solubilizer or surfactant may be present in an amount from about 0.1 wt % to about 20 wt %, such as from about 0.5 wt % to about 10 wt %.

The pharmaceutical composition may further include from about 0.1% to about 2.5% sodium chloride, or up to 10% of additional salts such as potassium chloride or sodium citrate.

The pharmaceutical composition may further include a preservative such as sodium benzoate. The preservative may be included in an amount from about 0.1% to about 3%.

The pharmaceutical composition may further include a humectant. The humectant may include any humectant suitable for topical applications, such as glycerine, urea, hyaluronic acid, salicylic acid, alpha hydroxy acids, propylene glycol, sorbitol, or a combination thereof. The humectant may be present in the pharmaceutical composition an amount from about 1 wt % to about 5 wt %.

The pharmaceutical composition may include a carrier. The carrier may include water for injection, a saline solution, a lipid, an emulsion, petrolatum, and the like.

When the pharmaceutical composition is formulated as an emulsion, ointment, paste, or cream, the composition may further include an emulsifier. The emulsifier may include cetostearyl alcohol, polysorbate 60 or 80 (Tween 60/80) or other nonionic surfactants, lecithin (phosphatidylcholine) extracted from eggs or soybeans, hydrogenated lecithin extracted form eggs or soybeans, beeswax, sodium stearoyl lactylate and similar compounds, olive oil products such as olive oil PEG-7 and PEG-6 esters, or any combination thereof.

The pharmaceutical composition may include an amino acid. Certain amino acid groups assist in protein or peptide solubility more than others. Aspartic acid, glutamic acid, and serine, in particular, have been noted for strong contributions to solubility. When antibiotic peptides and other drugs are relatively low in solubility-enhancing amino acids, achieving a solution, when desired, may require the addition of excipients that assist in solubilizing peptides, enhancing their suspension in aqueous solutions, or assist in their delivery via spray or nebulization. Such excipients may act as solvents, as surfactants, as stabilizers, etc.

In addition to the potential synergy between EDTA solutions and the antimicrobial, in some aspects, further synergy may also be realized with the presence of one or more additional active ingredients that are compatible with the conditions required for the EDTA-drug combination while also being effective in providing further synergy by, for example, weakening the protective material of a biofilm, interfering with microbial enzymes that contribute to drug resistance, or other effects that can enhance the efficacy of the drug-EDTA combination. Such additional active agents may include biofilm mitigation agents, enzyme inhibitors, inflammation control agents, nitric oxide (NO)-generating compounds, or other active agents.

The pharmaceutical composition may include one or more biofilm mitigation agents, such as N-acetyl cysteine, cysteine (including D-cysteine, L-cysteine, and combinations thereof), cysteine salts (including sodium cysteine salts, potassium cysteine salts, etc.), O-Acetylserine (OAS), glutathione, catechins such as epigallocatechin gallate (EGCG), ectoin, panthenol or panthothenic acid or derivatives thereof, (NO)-generating compounds, enzymes (including DNAse, lysozymes, proteinase, etc.), or any combination thereof.

Biofilm mitigation agents that enhance the biofilm mitigation ability of EDTA may be especially useful. For example, without wishing to be bound by theory, it is believed that one mechanism by which N-acetyl cysteine can undermine biofilms is through disrupting sulfide bonds. This mechanism may have synergy with EDTA's proposed mechanisms of biofilm mitigation through the chelation of certain cations. It is proposed that both may also inhibit enzymes associated with the virulence of biofilms and biofilm formation by altering the enzymes by attacking sulfide bonds and interfering with cations that are essential for some such enzymes. Thus, in one aspect, a composition and method for treating infection sites where biofilm is likely a threat may include a combination of EDTA, N-acetyl cysteine, and one or more antibiotics, wherein the application of the EDTA and N-acetyl cysteine (or EDTA alone) may weaken a biofilm and thereby enhance the ability of the antibiotic to control the targeted microbes. The biofilm mitigation agent may be present in the pharmaceutical composition in an amount from about 0.1 wt % to about 5 wt %. Infections that are likely to cause formation of biofilm includes Pseudomonas aeruginosa, Staphylococcus epidermidis, Escherichia coli, Klebsiella pneumoniae, Proteus mirabilis, Streptococcus viridans, Staphylococcus aureus, and Enterococcus faecalis.

The pharmaceutical composition may include enzyme inhibitors to undermine enzymes from various microbes that may contribute to drug resistance. Enzyme inhibitors may be provided to undermine enzymes from various microbes that may contribute to drug resistance. Stated another way, the enzyme inhibitor may be adapted to inhibit enzymes known to assist drug resistance relative to an antibiotic included in the composition. The enzyme inhibitors may be present in the pharmaceutical composition in an amount from about 0.1 wt % to about 5 wt %.

In some aspects, inflammation control agents such as ectoin may be applied in the lungs to reduce inflammation of airways and assist in healing while also serving as an effective antimicrobial against some bacteria such as Pseudomonas aeruginosa and Staphylococcus aureus.

In some aspects, NO-generating compounds may also be included to enhance the antimicrobial activity of the composition. The NO-generating compounds may include, for example, NONOates (also described as NONO-ates) in which the NONOate functional group —O—N═N—O—, a stabilized dimer of nitric oxide, can release NO over time in physiological settings or serve as a nitric oxide donor. Many parent amines can be used to prepare NONOates, giving rise to a variety of compounds such as spermine NONOate, DETA NONOate, PROLI NONOate, and EDTA NONOate. The pharmaceutical composition may include EDTA NONOate, spermine NONOate, DETA NONOate, PROLI NONOate, EDTA NONOate, the drug AB569, sodium nitrite or other acidified nitrites, polyamidoamines (including hyperbranched polyamidomines), polykanamycins (including hyperbranched polykanamycins), sodium nitroprusside, S-nitrosoglutathione, NO-releasing alginates, chitosan oligosaccharides, cephalosporin-based NO donor prodrugs (including cephalosporin-3′-diazeniumdiolates and diethylamino-cephalosporin-3′-diazeniumdiolate), or a combination thereof. The NO-generating compound may be present in the composition in an amount from about 1 wt % to about 20 wt %.

The synergy between EDTA and antimicrobials, with optional assistance from additional agents as described above, may be used to treat infections in wounds, on the skin, the sinuses, the mouth or throat, or other mucous membranes, including those in which biofilm is a significant problem.

Both the antimicrobial and the EDTA may be delivered via a topical ointment, spray, wet wipe, treated wound dressing, medicine dropper, or via a spray or an aerosol delivery device such as an inhaler, a nebulizer or atomizer, etc., from a mixture thereof or from two or more separate sources to provide the combination on the skin or other surface being treated, with or without time release of one or more agents.

The pharmaceutical composition may be in the form of a spray. The pharmaceutical composition may be packaged in a portable spray bottle, spray can, inhaler, and the like, which can generate a spray that may be applied to the treatment site.

For some antimicrobials, residing in contact with an alkaline EDTA solution for a prolonged period may hinder stability or efficacy. Thus, in some aspects, a kit is provided having two containers: a first container containing an antimicrobial composition such as a cream, wet wipe, or spray having a stable antimicrobial preparation at a suitable pH below 8.5, and a second container containing a pharmaceutical composition comprising an EDTA solution at a pH of 8.5 or higher such as from 9 to 11 or 9 to 10.5, wherein a two-part application may be made to the treatment site. The application of the antimicrobial composition and the pharmaceutical composition comprising the EDTA may be simultaneous, or one composition may be applied before the other. Preferably, the amount of time between application of both compositions is about 10 minutes or less.

To control the timing of EDTA activity or antimicrobial activity, one or more of the ingredients of the composition may be encapsulated with delayed release coatings, such that the encapsulated active ingredient is not active until after being exposed to a moist physiological system for a period of time. The coating may be adapted to dissolve or rupture in response to moisture, to a change in pH or temperature, to physical contact providing friction or compression, etc. Thus, in some aspects, EDTA and an alkaline buffering powder such as sodium bicarbonate may be encapsulated or embedded in the pharmaceutical composition, wherein the pharmaceutical composition is an ointment. The EDTA and buffering agent may gradually be released after being applied to the skin or other surfaces, providing a gradual biofilm inhibition effect and synergy with the antimicrobial. In other aspects, when the pharmaceutical composition is a gel, solution, or suspension, the microcapsules in a dry state may be blended with the pharmaceutical composition and immediately applied topically, after which the encapsulant may gradually rupture in response to the moisture and result in steady biofilm mitigation from the EDTA solution that is released from the capsules and the elevated pH that is maintained.

Encapsulation may be accomplished by spray drying coating systems, fluidized bed coating, extrusion in a lipid or polymer, coacervation, and the like. Polymers, gelatins, alginates, carrageenans, lipids, coacervates, etc., may be used to encapsulate any of the ingredients.

Further provided herein are methods for treating an infection, disease, or other condition in a subject in need thereof using the compositions described herein. The methods generally comprise contacting the treatment site with one or more pharmaceutical compositions as described herein, the treatment site may be sprayed, flushed, misted, wiped, spread, or daubed with the pharmaceutical composition described herein. In some embodiments, a pharmaceutical composition comprising EDTA may be applied and shortly thereafter (e.g., after from 30 seconds to 15 minutes, from 30 seconds to 5 minutes, from 20 seconds to 3 minutes, after 10 minutes, etc.), a composition comprising an antimicrobial compound is applied. The antimicrobial compound may be wiped, spread, daubed, poured, sprayed, flushed misted, or otherwise applied directly on the site or may first be applied to a wound dressing or other material that will contact the treatment site. In this way, an interaction between the EDTA and the antimicrobial can still be achieved with a reduced risk of instability or harm to the antimicrobial.

As used herein, the “treatment site” refers to the site on the subject wherein the infection, disease, or other condition to be treated is located. The pharmaceutical composition and optionally, one or more other compositions, are topically applied at the treatment site using one of the methods and apparatuses described herein. The treatment site may include, for example, a mucous membrane, the gums, the eyes, the sinuses, the throat, an open wound, an oral wound, damaged skin, a surgical wound, a site of a bacterial or fungal infection, etc.

The pharmaceutical composition may be applied once or may be applied multiple times to the treatment site. For example, the pharmaceutical composition may be applied five or more times per day, four or more times per day, three or more times per day, two or more times per day, once per day, once every two days, once every three days, and so on.

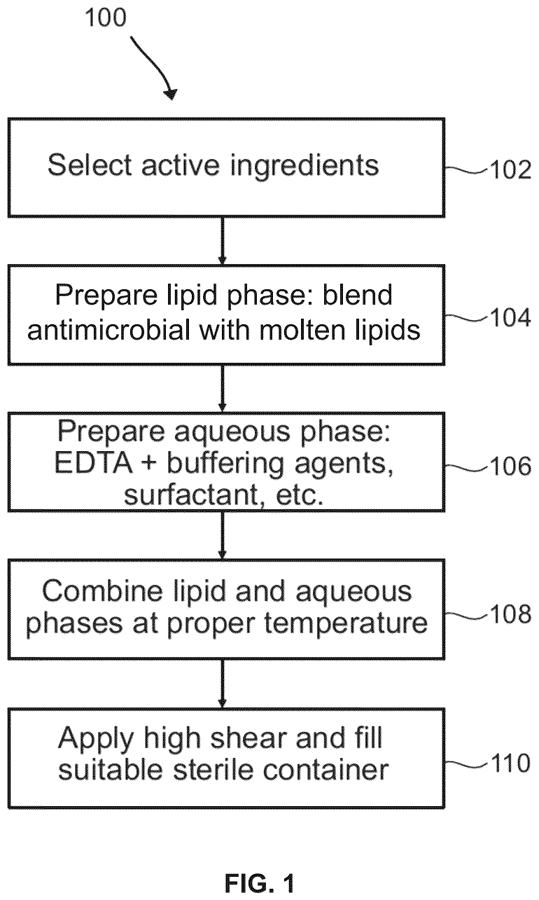

FIG. 1 depicts a method 100 of manufacturing a pharmaceutical composition of the present disclosure. In this aspect, the medicament is in the form of an oil-water emulsion that can function as a cream or ointment. In a first step 102, “select active ingredients,” suitable amounts of one or more antimicrobials are provided, as are correspondingly suitable amounts of lipids such as beeswax, cocoa butter, cetyl alcohol, etc., as well as EDTA, buffering agents or pH control agents (e.g., bicarbonates, citrates, carbonates, phosphates, etc.), emulsifiers, surfactants, humectants, biofilm control agents such as N-acetyl cysteine or ectoin, NO-generating agents, etc. Proposed formulations should be checked for compatibility of ingredients, pH, and manufacturing conditions.

In a second step 104, “prepare lipid phase: blend antimicrobial with molten lipids,” the lipids and compatible emulsifiers or other oil-phase ingredients (e.g., Vitamin E, etc.) are combined and heated and stirred to provide a uniform oil phase blend at a suitable temperature (e.g., 65° C., 70° C., 75° C., etc.) that will not damage the antimicrobials. This may include emulsifying waxes or other emulsifiers.

In a third step, 106, “prepare aqueous phase: EDTA+buffering agents, surfactant, etc.,” the aqueous phase ingredients are mixed and heated. (The emulsifiers may be best blended with the oil phase in the second step 104, but some emulsifiers may also be considered in the third step 106.)

A fourth step 108, “combine lipid and aqueous phases at the proper temperature,” the oil and water phases are blended at a suitable temperature, typically under stirring.

In a fifth step 110, “apply high shear and fill suitable sterile container,” the mixing of the two phases may be completed at high shear, and then the flowable emulsion may be used to fill containers such as flexible tubes or jars, which can then be labeled, tracked, and shipped for medical use.

In embodiments where the pharmaceutical composition is a spray, the antimicrobial, the EDTA, and any of the other ingredients described above may be combined in a carrier, such as an aqueous carrier, prior to application.

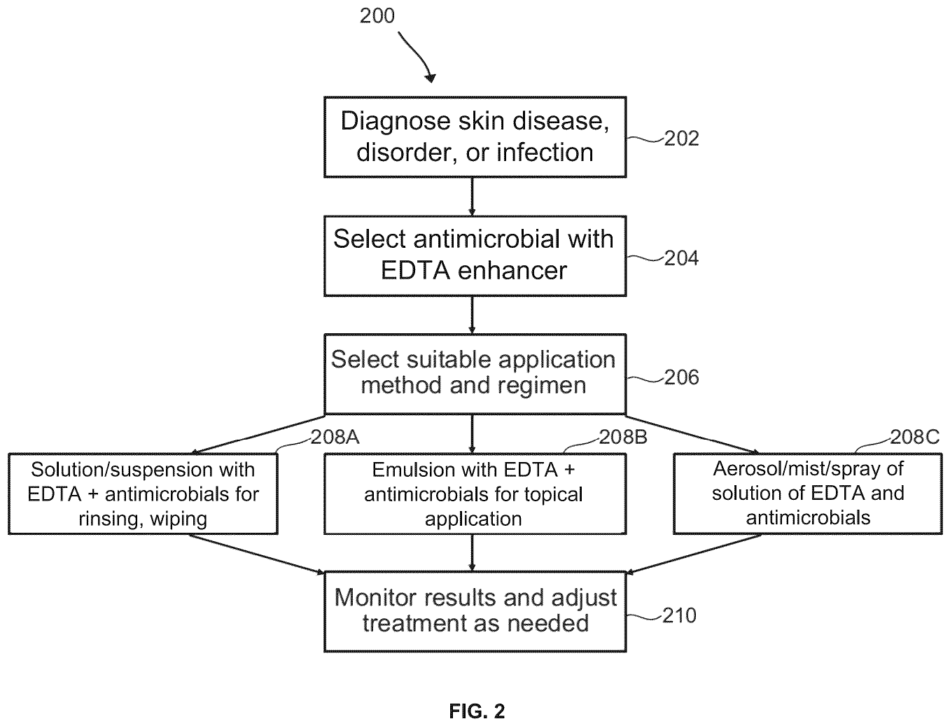

FIG. 2 depicts a method for applying EDTA and an antimicrobial topically to a treatment site of a subject in need thereof. In a first step 202, “diagnose skin infection,” a physician determines the nature of an infection or wound site and assesses if a microbial infection is present in need of antibiotic treatment.

In light of risks of promoting or creating disease-resistant organisms, or in light of the risk that a disease-resistant organism could be active, or in light of the risk of a superinfection that might be caused by use of the antimicrobial under consideration, in a second step 204, “select antibiotic system with EDTA enhancer,” the physician selects a suitable drug such and EDTA system to reduce the risks and optimize the patient outcome, considering drug efficacy, solubility, interactions with EDTA, etc. Compatibility of available EDTA concentrations and the other ingredients with the infected site may need to be considered. For example, the physician may determine that a sulfone is needed, coupled with EDTA at a concentration of at least 1% to reduce risks of superinfection or disease-resistant organisms, but that it may need to first be sprayed periodically.

In a third step 206, “select suitable application method and regimen,” the physician may determine, for example, that the infected site is too sensitive to use a wipe or to directly apply a cream and that initially a spray should be applied, but then after perhaps after 3 days a cream or ointment with a lower level of EDTA may be applied.

The physician may then make a choice between three available options for the antimicrobial of choice: (1) solution/suspension with EDTA+antibiotics for rinsing, wiping, 208A; (2) emulsion with EDTA+antibiotics for topical application, 208B; and (3) aerosol/mist/spray of solution of EDTA and antibiotics, 208C. The physician may select spraying, part of choice (3) for initial treatment, to be followed by application of an ointment according to choice (1).

Treatment is then initiated and, in the last step shown 210, “monitor results and adjust treatment as needed,” the progress of the patient is monitored, and the treatment regime is adjusted as needed.

Several aspects of the disclosure may now be considered. In a first aspect, a method is provided for topically treating an infection site in a patient, comprising applying to the infection site a pharmaceutically effective dose of an antibiotic susceptible to drug-resistant organisms and applying to the infection site an aqueous medium comprising an effective amount of EDTA at a pH from 8.5 to 11, wherein the method has a reduced risk of creating disease-resistant organisms relative to an otherwise identical method without the application of EDTA.

In a second aspect, the method of Aspect 1 is provided, wherein the aqueous medium is dispersed in an oil-water emulsion that comprises the antibiotic.

In a third aspect, the method of Aspect 1 or 2 is provided, wherein the antibiotic is dissolved or suspended in the aqueous medium, and wherein application of the aqueous medium to the infection site comprises spraying or aerosolizing the aqueous medium to deliver droplets to the site.

In a fourth aspect, the method of any of the preceding aspects is provided, wherein the infection is a skin or surgical wound, and the aqueous medium further comprises at least one of the groups consisting of a NO-generating compound, N-acetyl cysteine, ectoin, and an enzyme inhibitor adapted to inhibit enzymes known to assist drug resistance relative to the antibiotic.

In fifth aspect, the method of any of the preceding aspects is provided, wherein the antibiotic is selected from the group consisting of amikacin, ampicillin, anidulafungin, azidamfenicol, bacitracin, capreomycin, caspofungin, cefazolin, cefepime, cefotaxime, ceftriaxone, cephalosporins, chloramphenicol, ciprofloxacin, clarithromycin, clindamycin, colistin, contezolid amoxicillin, cyclosporine, dalfopristin, daptomycin, doxycycline, enviomycin, eravacycline, ertapenem, erythromycin aztreonam, florfenicol, fluconazole, gentamicin, gramicidin, imipenem, isavuconazole, itraconazole, iturin, levofloxacin, linezolid, meropenem, methicillin, metronidazole, micafungin, minocycline, moxifloxacin, neomycin, nisin, omadacycline, penicillin, polymyxin B, posaconazole, quinupristin, ramoplanin, rifabutin, rifalazil sulfamethoxazole, rifampin, rifapentine, streptomycin, sulfacetamide, sulfadiazine, sulfadoxine tetracycline, sulfasalazine, sulfathiazole, sulfisoxazole, sulfonamides, surfactin, teicoplanin, thiamphenicol, tigecycline, tobramycin doripenem, tyrothricin azithromycin, vancomycin, viomycin, and voriconazole.

In a sixth aspect, the method of any of the preceding aspects is provided, wherein the infection site is in the sinuses, nose, oral cavity, or throat.

In a seventh aspect, a topical therapeutic composition is provided that is effective at reducing a microbial infection from an identified microbe, the topical therapeutic composition comprising an antibiotic known to be at risk from disease-resistant organisms or at risk of promoting disease-resistant organisms, comprising an effective amount of at least one antibiotic and comprising at least 0.5 wt % EDTA, or at least 1 wt %, 2 wt %, 3 wt %, 4 wt %, 5 wt %, 8 wt %, 10 wt % or 12 wt % EDTA such as from 1 wt % to 15 wt % EDTA, wherein the topical therapeutic composition has a pH of 8.5 to 11 and wherein the topical therapeutic composition has a reduced risk of promoting disease-resistant organisms than an otherwise identical composition lacking the EDTA.

In an eighth aspect, a method is provided for treating an infection in a patient at reduced risk of creating disease-resistant organisms, including providing a pharmaceutically effective dose of an antibiotic susceptible to drug-resistant organisms provided in an aqueous medium comprising an effective amount of EDTA at a pH from 8.5 to 11, and topically applying the aqueous medium to an infection site.

In a ninth aspect, a method for topically treating a biofilm on a physiological surface is provided, comprising treating the biofilm with a solution of EDTA in an alkaline medium (e.g., pH of 8.5 or higher) and applying a suitable antibiotic. A synergistic biofilm control agent may be combined with the EDTA, such as N-acetyl cysteine, a cysteine salt, etc., to further undermine the biofilm and make the antibiotic more effective.

In other aspects, lipids are combined with the EDTA and antibiotic solution to prepare liposomes. This can be done with freeze-drying, spray drying, sonication, reverse phase evaporation, etc., and the liposomes can be dispersed in liquid again and delivered by a spray, droplets, a nebulizer, a brush applicator, etc. Nanoemulsions may also be used. The lipids may include lipids such as beeswax, cocoa butter, cetyl alcohol, cholesterol, distearoyl phosphotidyl-glycerol, cholesteryl sulphate, and combinations thereof. In such aspects, the lipids may be present in the pharmaceutical composition in an amount from about 0.1 wt % to about 50 wt %. Several further details of some aspects will now be considered.

### DETAILED EXEMPLARY ASPECTS

In a first prophetic example, amphotericin B (AMB) is used to prepare a dermatological ointment in a lipid carrier with hydrogenated phosphatidylcholine:cholesterol:distearoyl phosphotidyl-glycerol:AMB in ratio of 2:1:0.8:1, and blended with 30% by weight of a solution having 2% EDTA at a pH of 9.5, with a suitable emulsifier.

In a second prophetic example, a dermatological wound-care ointment is prepared from equal parts cholesteryl sulphate and AMB. In a molten state at 65° C., 100 parts of the mixture are blended with 30 parts 3% EDTA solution at a pH of 8.6 to 9.5 with 0.5% ectoin, 1.3% saline, 0.5% polysorbate 80, 2% lecithin, and 1% of a suitable preservative, and the ingredients are blended in a mixer to form an emulsion.

In a third prophetic example, a triple antibiotic ointment is prepared. In a base of white petrolatum, the following antibiotics are blended at the indicated dose per gram of petrolatum: bacitracin zinc USP, 500 units; neomycin, 3.5 mg, polymyxin B sulfate USP, 10,000 units. The mixture is then melted and blended at high shear with 10% by weight of EDTA at 6% concentration at a pH of 9.7 in a carbonate-bicarbonate buffer system, with 0.2% of a suitable surfactant and 1% lecithin to form a cream.

In a fourth prophetic example, daptomycin is used for treating nasal infections in a mammal from a specific Gram-positive bacterium. It is provided in a nebulizer liquid at a concentration of 100% above the MIC with EDTA at a concentration of 2% in a hypertrophic saline solution at 3% concentration and a pH of 10. A carbonate-bicarbonate buffer system is included. 0.2% polysorbate 80 is added as a surfactant. An ultrasonic mesh nebulizer is used to deliver the medicament.

In a fifth prophetic example, a wound care ointment is provided by forming a cream with 4% cephalexin powder, 40% white beeswax, 15% cetyl alcohol, 6% propylene glycol, 2% sodium lauryl sulfate, and 20% water of an aqueous solution having 6% EDTA at a pH of 9.6 with a suitable emulsifier.

In a sixth prophetic example, a patient is diagnosed with a nasal infection from gram-negative aerobic pathogens, likely including Pseudomonas aeruginosa, for which aztreonam is deemed suitable. To reduce the risk of superinfection from gram-positive pathogens, for which aztreonam has little effect, the antibiotic is prepared in solution with EDTA buffered with pharmaceutically acceptable agents such as alkaline glycine to give a pH of 10.5. The EDTA concentration is 2%, and the aztreonam concentration is 90% above the MIC for Pseudomonas aeruginosa. Treatment is provided periodically with a handheld nasal spray bottle.

### EXAMPLES

Strains of E. coli and S. epidermidis were contacted with compositions that included tetrasodium EDTA (T-EDTA) and tetracycline. Microbroth dilution in 96-well microtiter plates was performed on the E. coli and S. epidermidis strains. 200 μL of two-fold dilutions of tetrasodium EDTA (or tetracycline) were prepared in Mueller-Hinton or Mueller-Hinton II broth. The plate contained decreasing concentrations of tetrasodium EDTA (4%-0.03%) or tetracycline (512-0.5 μg/mL) in columns 1-11. Then, 10 μL of standardized cell suspension was added to each well. The microtiter plates were incubated at 37° C. for 24 h and the results were analyzed. Each test was performed in duplicate. The MIC was determined as the lowest concentration where no growth was observed, but the adjacent wells had growth as determined by optical density. The MIC values of T-EDTA and tetracycline were determined in isolation and are provided in Table 1.

TABLE 1
MIC in Isolation

| | MIC in Isolation | |
|---|---|---|
| Strains | T-EDTA (wt %) | Tetracycline (μg/mL) |
| E. Coli HC1-R2A-1 | 1 | 128 |
| S. epidermidis HC3-CA-1 | 0.0625 | 8 |

At the end of the MIC experiment, 5 μL aliquots were removed from each well in the 96-well MIC plates and used to inoculate another microtiter plate that was filled with Mueller-Hinton media. This new microtiter plate was incubated at 37° C. and growth was quantified after 24 h by measuring the optical density in the wells. The MBC was determined to be the lowest concentration of t-EDTA or Tetracycline where no growth was observed. The MBC values of T-EDTA and tetracycline were determined in isolation and are provided in Table 2.

TABLE 2
MBC in Isolation

| | MBC in Isolation | |
|---|---|---|
| Strains | T-EDTA (wt %) | Tetracycline (μg/mL) |
| E. Coli HC1-R2A-1 | 1 | 512 |
| S. epidermidis HC3-CA-1 | 4 | 8 |

Next, compositions were prepared that included a combination of T-EDTA and tetracycline. The interaction of tetrasodium EDTA with tetracycline was investigated by the checkerboard titration method using microbroth dilution in 96-well microtiter plates. 200 μL of two-fold dilutions of tetrasodium EDTA and tetracycline were prepared in MH or MH II broth. The plate contained decreasing concentrations of tetracycline (256 μg/mL-0.5 μg/mL) in columns 1-10 and decreasing concentrations of tetrasodium EDTA (2%-0.015%) in rows A-H. Then, 10 μL of standardized cell suspension was added to each well. Microtiter plates were incubated at 37° C. for 24 h and the results were analyzed. Each test included a growth control without addition of any antimicrobials, as well as an uninoculated control column to ensure there was no contamination in the plate. The combination MIC was taken as the concentration where no growth was observed by optical density but the wells immediately adjacent had growth. To determine the MBC, 5 μL aliquots were removed from each well in the 96-well MIC plates above and used to inoculate a new microtiter plate that was filled with Mueller-Hinton media. This new microtiter plate was incubated at 37° C. for 24 h and growth was quantified by measuring the optical density in the wells. The MBC was determined to be the lowest combination of t-EDTA and Tetracycline where no growth was observed, but the wells immediately adjacent had growth. The MIC and MBC were once again determined with the combinations, and the results are shown in Tables 3 and 4, respectively.

The potential interaction of the two antimicrobials was determined by the FICI (fractional inhibitory concentration index) and FBCI (fractional bactericidal concentration index). Note: tetracycline=tet.

$$FICI = \frac{MIC of tet . in combination with EDTA}{MIC of tet . alone} + \frac{MIC of EDTA in combination with tet .}{MIC of EDTA alone}$$

$$FBCI = \frac{MBC of tet . in combination with EDTA}{MBC of tet . alone} + \frac{MBC of EDTA in combination with tet .}{MBC of EDTA alone}$$

Deviations from FICI or FBCI=1 indicate interactions: FICI>1 shows negative interaction, while FICI<1 indicates a positive one. Practical limits define synergy as FICI≤0.5 and antagonism as FICI≥4.

TABLE 3
MIC in Combination

| | MIC in Combination | | |
|---|---|---|---|
| | T-EDTA | Tetracycline | |
| Strains | (wt %) | (μg/mL) | FICI |
| E. Coli HC1-R2A-1 | 0.25 | 8 | 0.3 |
| | | | (Synergy) |
| S. epidermidis | 0.015 | 0.5 | 0.3 |
| HC3-CA-1 | | | (Synergy) |

TABLE 2
MBC in Combination

| | MBC in Combination | | |
|---|---|---|---|
| | T-EDTA | Tetracycline | |
| Strains | (wt %) | (μg/mL) | FBCI |
| E. Coli HC1-R2A-1 | 0.25 | 8 | 0.25 |
| | | | (Synergy) |
| S. epidermidis | 0.25 | 1 | 0.18 |
| HC3-CA-1 | | | (Synergy) |

The results clearly show a synergistic effect from the combination of the antimicrobial (tetracycline) and the EDTA by reducing the MIC and MBC of both the EDTA and the antimicrobial.

The term “about” is used herein to mean approximately, roughly, around, or in the region of. When the term “about” is used in conjunction with a numerical range, it modifies that range by extending the boundaries above and below the numerical values set forth. The term “about” is used herein to modify a numerical value above and below the stated value by a variance of 20 percent up or down (higher or lower), preferably 15 percent, more preferably 10 percent, and most preferably 5 percent.

When introducing elements of aspects of the invention or aspects thereof, the articles “a,” “an,” “the,” and “said” are intended to mean that there are one or more of the elements. The terms “comprising,” “including,” and “having” are inclusive and mean that there may be additional elements other than the listed elements.

The term “therapeutically effective amount” means an amount of active ingredient(s) effective to prevent, slow, alleviate or ameliorate symptoms of a disorder or condition, or to prolong the survival of the subject being treated. Determination of a therapeutically effective amount of an ingredient is within the capability of those skilled in the art, especially in light of the detailed disclosure provided herein.

The term “pharmaceutical composition” is defined as a chemical or a biological compound or substance, or a mixture or combination of two or more such compounds or substances, intended for use in the medical diagnosis, cure, treatment, or prevention of disease or pathology. As used herein “pharmaceutical composition” and “medicament” may be used interchangeably.

The term “treat” or “treatment” refers to administration of an active pharmaceutical ingredient to a subject aimed at preventing, alleviating, and/or curing a medical condition, disease, or disorder.

Having described aspects of the invention in detail, it will be apparent that modifications and variations are possible without departing from the scope of aspects of the invention as defined in the appended claims. As various changes could be made in the above compositions, products, and methods without departing from the scope of aspects of the invention, it is intended that all matter contained in the above description shall be interpreted as illustrative and not in a limiting sense.

While the foregoing makes reference to particular illustrative embodiments, these examples should not be construed as limitations. The inventive system, methods, and products can be adapted for other uses or forms not explicitly listed above and can be modified in numerous ways within the spirit of the present disclosure. Thus, the present invention is not limited to the disclosed aspects but is to be accorded the widest scope consistent with the claims below.

We claim:

1. A method for topically treating a treatment site in a subject in need thereof comprising contacting the treatment site with a pharmaceutical composition comprising:
a therapeutically effective amount of an antimicrobial susceptible to drug-resistant organisms; and
a therapeutically effective amount of EDTA,
wherein the pharmaceutical composition has a pH from 8.5 to 11, wherein the method has a reduced risk of creating disease-resistant organisms relative to an otherwise identical method without the application of EDTA.

2. The method of claim 1, wherein the pharmaceutical composition comprises an oil-water emulsion that comprises the antimicrobial.

3. The method of claim 1, wherein the antimicrobial is dissolved or suspended in an aqueous medium, and wherein contacting the treatment site with the pharmaceutical composition comprises spraying or aerosolizing the pharmaceutical composition.

4. The method of claim 1, wherein the pharmaceutical composition further comprises at least one of the group consisting of an nitric oxide (NO)-generating compound, N-acetyl cysteine, ectoin, an enzyme inhibitor adapted to inhibit enzymes known to assist drug resistance relative to the antibiotic, and any combination thereof.

5. The method of claim 1, wherein the treatment site is skin or a surgical wound.

6. The method of claim 1, wherein the antimicrobial is selected from the group consisting of amikacin, ampicillin, anidulafungin, azidamfenicol, bacitracin, capreomycin, caspofungin, cefazolin, cefepime, cefotaxime, ceftriaxone, cephalosporins, chloramphenicol, ciprofloxacin, clarithromycin, clindamycin, colistin, contezolid amoxicillin, cyclosporine, dalfopristin, daptomycin, doxycycline, enviomycin, eravacycline, ertapenem, erythromycin aztreonam, florfenicol, fluconazole, gentamicin, gramicidin, imipenem, isavuconazole, itraconazole, iturin, levofloxacin, linezolid, meropenem, methicillin, metronidazole, micafungin, minocycline, moxifloxacin, neomycin, nisin, omadacycline, penicillin, polymyxin B, posaconazole, quinupristin, ramoplanin, rifabutin, rifalazil sulfamethoxazole, rifampin, rifapentine, streptomycin, sulfacetamide, sulfadiazine, sulfadoxine tetracycline, sulfasalazine, sulfathiazole, sulfisoxazole, sulfonamides, surfactin, teicoplanin, thiamphenicol, tigecycline, tobramycin doripenem, tyrothricin azithromycin, vancomycin, viomycin, voriconazole, and any combination thereof.

7. The method of claim 1, wherein treatment site is the sinuses, nose, oral cavity or throat.

8. A method for topically treating a treatment site in a subject in need thereof comprising:
contacting the treatment site with a first pharmaceutical composition comprising a therapeutically effective amount of an antimicrobial susceptible to drug-resistant organisms; and
contacting the treatment site with a second pharmaceutical composition comprising a therapeutically effective amount of EDTA,
wherein the second pharmaceutical composition has a pH from 8.5 to 11, and
wherein the method has a reduced risk of creating disease-resistant organisms relative to an otherwise identical method without the application of EDTA.

9. The method of claim 8, wherein the steps of contacting the treatment site with the first pharmaceutical composition and contacting the treatment site with the second pharmaceutical composition are performed simultaneously.

10. The method of claim 8, wherein the steps of contacting the treatment site with the first pharmaceutical composition and contacting the treatment site with the second pharmaceutical composition are performed within about 10 minutes of another.

11. A topical pharmaceutical composition effective at reducing a microbial infection from a microbe, the topical pharmaceutical composition comprising:
an antimicrobial known to be at risk from disease-resistant organisms or at risk of promoting disease-resistant organisms; and
at least 0.1% EDTA,
wherein the topical therapeutic composition has a pH of 8.5 to 11 and wherein the topical therapeutic composition has a reduced risk of promoting disease-resistant organisms than an otherwise identical composition lacking the EDTA.

12. The composition of claim 11, wherein the antimicrobial comprises an antibiotic or an antifungal.

13. The composition of claim 12, wherein the antibiotic is selected from the group consisting of amikacin, ampicillin, anidulafungin, azidamfenicol, bacitracin, capreomycin, caspofungin, cefazolin, cefepime, cefotaxime, ceftriaxone, cephalosporins, chloramphenicol, ciprofloxacin, clarithromycin, clindamycin, colistin, contezolid amoxicillin, cyclosporine, dalfopristin, daptomycin, doxycycline, enviomycin, eravacycline, ertapenem, erythromycin aztreonam, florfenicol, fluconazole, gentamicin, gramicidin, imipenem, isavuconazole, itraconazole, iturin, levofloxacin, linezolid, meropenem, methicillin, metronidazole, micafungin, minocycline, moxifloxacin, neomycin, nisin, omadacycline, penicillin, polymyxin B, posaconazole, quinupristin, ramoplanin, rifabutin, rifalazil sulfamethoxazole, rifampin, rifapentine, streptomycin, sulfacetamide, sulfadiazine, sulfadoxine tetracycline, sulfasalazine, sulfathiazole, sulfisoxazole, sulfonamides, surfactin, teicoplanin, thiamphenicol, tigecycline, tobramycin doripenem, tyrothricin azithromycin, vancomycin, viomycin, voriconazole, and combinations thereof.

14. The composition of claim 11, further comprising a mucolytic agent, a biofilm mitigation agent, an enzyme inhibitor, a NO-generating compound, or a combination thereof.

15. The composition of claim 11 further comprising a buffer, a solubilizer, a surfactant, a salt, an amino acid, one or more lipids, or a combination thereof.

16. The composition of claim 13, wherein the EDTA is present in a concentration of at least about 0.5 wt %.

17. The composition of claim 13, wherein the EDTA is present in a concentration from about 0.1 wt % to about 15 wt %.

18. The composition of claim 13, wherein the composition comprises an aqueous phase and a lipid phase, wherein the EDTA is in the aqueous phase and the antimicrobial is in the lipid phase.

19. The composition of claim 13, wherein the composition is a cream, an ointment, a spray, a solution, a colloidal suspension, a slurry, a paste, a spray, an aerosol, droplets, an emulsion, or a flush.

20. A kit comprising:
a first container including a first pharmaceutical composition suitable for topical application comprising EDTA at a pH of greater than 8.5; and
a second container including a second pharmaceutical composition suitable for topical application comprising an antimicrobial.
